(19) United States
(12) Patent Application Publication
(10) Pub. No.: US 2023/0390775 A1
(43) Pub. Date: Dec. 7, 2023

(54) Method and Apparatus for Dual Use Microbiology Test Card

(71) Applicant: Jonathan N. Roth, Goshen, IN (US)

(72) Inventor: Jonathan N. Roth, Goshen, IN (US)

(21) Appl. No.: 17/803,958

(22) Filed: Feb. 6, 2023

Related U.S. Application Data
(63) Continuation-in-part of application No. 17/803,380, filed on Jun. 6, 2022.

(51) Int. Cl. B01L 3/00; C12Q 1/04; C12M 1/12

(52) U.S. Cl. CPC B01L 3/5085; C12Q 1/04; C12M 23/04; B01L 3/5023; B01L 2300/0816; B01L 2200/16; B01L 2300/069

(57) ABSTRACT

A test card is provided for separately but simultaneously or sequentially detecting the existence and/or identity of different samples of microorganisms, having three layers which allow total separation, growth, and testing of different test samples which require chemically different growth media or replicative simultaneous testing.

The present application is a continuation-in-part of the patent application for the invention disclosed in U.S. patent application Ser. No. 17/803,380, filed on Jun. 6, 2022, entitled Improved Microbiology Test Card. The disclosure of that prior patent application is incorporated herein by reference.

### BACKGROUND OF THE INVENTION

The present invention relates generally to methods, apparatus and processes for preparing and examining samples of micro-organisms, and, more particularly, for detecting the presence of harmful bacteria and other micro-organisms.

Various prior devices are known for detecting the presence of coliforms, and other bacteria, by depositing a fluidic test sample onto a card, securing that sample in place on the card in the presence of a growth medium by means of a transparent top cover or film, and then allowing the organism to grow for a period of time (with or without the use of a supplemental incubation environment) sufficient for the organism and/or its growth bi-products to react with a chemical medium to produce a visual indicia indicating its presence. Often, this visual indicia is a color or chromogenic change observable on the card directly and/or in the presence of ultra violet or fluorogenic light. In some prior devices, the cards may use various color indicia for verification of the presence of a particular micro-organism or to simultaneously detect multiple different types of micro-organisms.

However, such prior devices have exhibited certain difficulties and/or disadvantages. For example, for various reasons, sometimes relating to the card structures, and sometimes to the specific growth media and visual indicator chemistry, for example, certain types of prior test cards have provided inconsistent, false positive, and/or false negative test results. Also, certain types of prior test cards were not as well suited to automatic detection devices and/or reliable optical scanning as would be desired. Further, such cards sometimes required more expensive production methods than would be desirable, and/or have an unduly short shelf life pending use. In addition, certain prior test cards were not as adaptable to alternative uses as would be desired.

In addition, numerous prior microbiology test card products have been developed which purport to yield faster results and be easier to use, less expensive, more biodegradable, more accurate and more portable. However, often those products fill only a given niche, as for one specific, or closely related species or genera of microbes, especially as per a single test card product. Such prior products cannot, for example, efficiently grow and clearly differentiate more than one specific, non-closely related species or genera of microbes on a single test card, and can be unreliable across different or various species of microorganisms.

### Objectives of the Invention:

Accordingly, a primary objective of the present invention is to provide improved test cards for detection of micro-organisms. These improvements include providing microbiology test cards which:
a. have a less overall cost of manufacture, longevity, and use,
b. increase the economy and/or versatility of card use,
c. provide more reliable and consistent test results,
d. accommodate testing of different micro-organism types on a single card,
e. increase user safety and convenience, and
f. have less bulk for waste and are more readily biodegradable.

### SUMMARY OF THE INVENTION

These and other objectives of the present invention are achieved by the provision of a card having a three layer structure which is used to provide total separation and growth of different microbiological entities which require different chemical ingredients, hence avoiding confusing mixture of the target entities. This three layer structure includes a central layer of opaque, waterproof material with sufficient stiffness and composition to both avoid difficulty in handling and any leakage. Two additional, top and bottom layers of non-toxic, transparent, gas permeable, non-water absorbent, printable, flexible plastic are secured to each side of the central layer by a non-toxic, clear adhesive film which is applied to the entire surface of these two layers which abuts the central layer, but which is in direct contact with the central layer along only a peripheral side strip of the top and bottom layers. The remainder of the adhesive film on the top and bottom layers is covered with an amount of growth media formulations which adhere to the adhesive and are specific for diverse microbiological organisms to be tested. Thus, a different growth media can be used with the top layer than with the bottom layer.

Using this structure, two different test samples (preferably fluidic) of microbiological organisms can be applied to the same card, between the top layer and the central layer and between the bottom layer and the central layer, in a conventional manner. Testing for specific different types of organisms can be simultaneous if the physical factors, such as incubation temperature or gas type are within the acceptable range for both organisms, or subsequently if those physical factors differ significantly. To facilitate manual and/or automated counting of visual indications of micro-organisms, a black ink grid system can be applied to the top and bottom layers which does not diffuse in the presence of the fluidic sample and/or the media, but at the same time which readily is removable or omitted from the base when removal is desired.

Alternatively, the same type of microbiological organism (or multiple samples from the same microorganism pool, type, or environment) can be applied to both sides of the card for simultaneous testing using the same growth media and conditions (as a replication or verification test, for example), or using different growth media and/or conditions on each side of the card.

Other objects, advantages, and novel features of the present invention will become readily apparent from the following drawings and detailed description of certain preferred and alternative embodiments.

### BRIEF DESCRIPTION OF THE DRAWINGS

FIG. 1 is a top view of a test card incorporating the present invention.

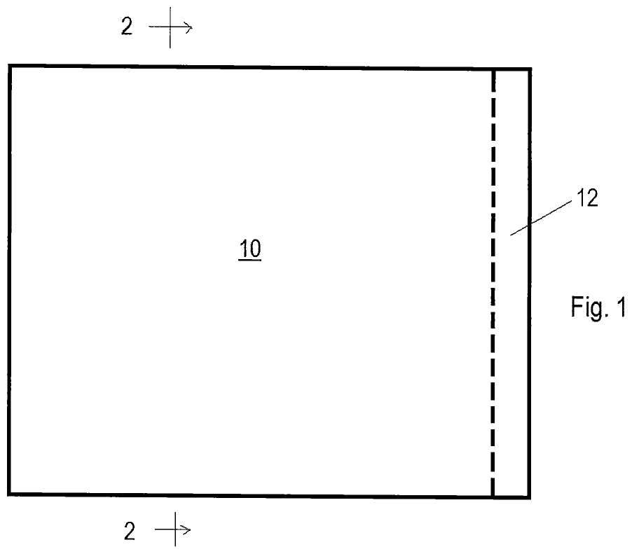

FIG. 2 is an exploded, cross-sectional view of the test card of FIG. 1 from left to right, taken along line 2-2.

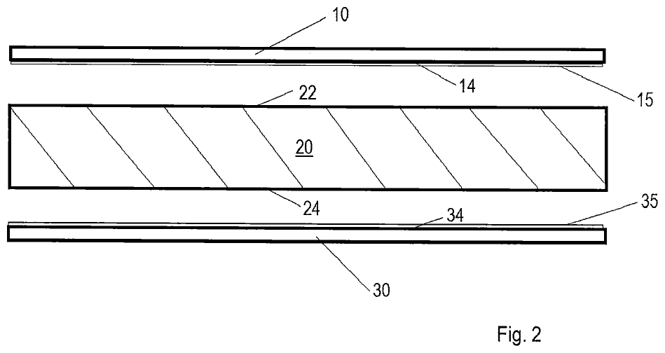

### DETAILED DESCRIPTION OF PREFERRED EMBODIMENTS

The figures show an embodiment of the present invention, particularly as applied to a test card for receiving two different fluidic test samples. The figures are not precisely to scale from one to the next.

The following numbered features of the test card are illustrated in the drawings:
a top layer 10, having a generally rectangular top configuration, and
   a peripheral side strip 12 along a side of the top layer 10,
   a bottom surface 14 covered with an adhesive film,
   a first growth media 15 covering the adhesive film on bottom surface 14, except in the area of peripheral side strip 12,
a central or middle layer 20, having a top-viewed configuration matching that of top layer 10, and
   a top surface 22,
   a bottom surface 24, and
a bottom layer 30, having a top-viewed configuration matching that of top layer 10, and
   a top surface 32,
   a bottom surface 34 covered with adhesive film,
   a second growth media 35 covering the adhesive film, except in the area of bottom layer 30 corresponding to the location of the peripheral side strip 12.

In one preferred embodiment, the test card can be formed as a 3 inch×3 inch square card, when viewed from the top of the card. However, as desired for particular applications and automated test equipment, other configurations can be used. In general, however, such as where a rectangular card is used, the top, central, and bottom layers preferably have the same shape and dimensions when viewed from the top of the completed card. The top and bottom layers are joined to the central layer, for example, by the adhesive film along one of the peripheral sides of each of the top and bottom layers, such that the top and bottom layers can each be flexed back and away from the central layer in order to permit the test sample to be disposed therebetween in a conventional manner of application, and then flexed down onto the central layer once the test sample is in place and growth testing is to begin.

The growth medium, which may be selected to be of different formulations for each of the top and bottom layers (according to the particular type of micro-organism to be tested for), is preferably applied as a powder on top of the adhesive film formed on bottom surfaces 14 and 34 of the top and bottom layers, except in the areas of those layers where the adhesive or a tape hinge or the like may be used for attachment of those layers to the central layer (preferably, for example, in the areas of peripheral strips 12). For example, the top and bottom layers can each be formed for manufacturing from a plastic strip of material with an adhesive film applied to one side thereof and then covered by peel-away paper. As each such layer is then cut to the desired shape for assembling the test card, one side of that layer has that paper scored to a width of ⅜ inch. Thereafter, the paper is peeled off the card to expose the adhesive film over the entire bottom surface of that layer except the area under the ⅜ inch wide peripheral side strip 12. After that exposed portion of the adhesive film receives and is covered by the growth media, the remaining ⅜ inch wide paper strip is peeled away to expose the remaining adhesive film, and then that layer is attached to the central layer via that remaining adhesive film, or by other means, such as tape or a hinged-like device.

In especially preferred embodiments, the top and bottom layers are each formed from clear polystyrene, polypropylene, or like plastic material, such as commercially available acetate film, of 2 mil in thickness. It is important that these layers be transparent and gas permeable so that microbial growth can be readily observed and that sufficient gas is accessible therethrough to the test sample in order to permit microbial growth in the desired test time period. However, it is also important that these layers are not water absorbent, at least to a degree which affects the microbial growth or stability of the test sample or creates leakage from the card during testing, while at the same time not be so gas permeable as to allow the test sample to dry out too quickly. It is further important that the top and bottom layers be sufficiently flexible to allow that layer to be bent away (without permanent deformation) from the central layer when the test sample is to be applied to the card. It is also desirable for the top and bottom layers to be formed from material which will receive and retain without unintended smearing or smudging a printed pattern thereon, such as a grid pattern, so as to facilitate manual or automated microbial colony counting.

In preferred embodiments, the adhesive film used on the top and bottom layers is transparent and completely non-toxic to the microbiological organisms being tested for. For example, commercially available Technicoat brand AT333 adhesive has been found to be a suitable adhesive when testing for Salmonella, E. coli, Enterococcus, and Listeria.

Growth media 15 and 35 may not be of the same formulation, and are instead selected according to conventional information to support growth of the particular microorganisms being tested for. Also, in preferred embodiments, if the growth media selected for a given microbial test is based upon or incorporates guar gum, that gum should be sterilized against viable bacteria, mold, or bacterial spores prior to or upon application to the adhesive film, since “food grade” guar gum powder commonly imported into the United States within recent years has been subject to only limited testing (for Salmonella and E. coli, for example) and have routinely contained many contaminants.

In preferred embodiments, the central or middle layer is formed from an opaque and waterproof material, such as Hop-Syn brand synthetic paper, of 10 mil thickness. This will prevent migration of microorganisms between surfaces 22 and 24 during and after testing. Care should be given in selection of the material, weight and thickness of other embodiments of the central layer that in simultaneous testing of test samples adjacent surfaces 22 and 24, growth of the lower test sample is not mechanically or physically inhibited.

### Briefly, these Elements Interact as Follows

The first sample of microbial material to be tested is, preferably, but not necessarily, entrained in a fluid such as water, and is then deposited between the top layer and the central layer, and then the top layer is folded or lowered back down onto the central layer to disperse and retain the first sample between those layers. Thereafter, the second sample, of different microbial material, to be tested is similarly deposited between the bottom layer and the central layer, and then the bottom layer is folded or lowered back down onto the central layer to disperse and retain the second sample between those layers. If the two samples are to be tested simultaneously in the same test/growth equipment (such as for precise and rapid comparative testing, and without confusion between different cards), the card is then inserted into and retained within the incubation or other growth environment in a conventional manner. If the two samples are to be tested sequentially, the second sample is preferably not applied to the card until after testing of the first sample is completed.

Although the present invention has been shown and described herein with respect to certain preferred embodiments and alternative configurations, those were by way of illustration and example only. For example, in especially preferred embodiments, the card has been shown in substantially square formats, but the present invention contemplates other card shapes, such as hexagonal or other rectangular formats. Also, while the card is described for use in connection with fluidic test samples, in some embodiments, solid test samples can be placed on the base portion instead, or the cover portion can be left open for a time to permit air-borne microbial particles, mold spores, and like items to be deposited on the base portion and/or media. Further, the present invention can be used to allow both sides of the card to test the same type of fluidic sample are a replication to verify the results for that type of sample, or to test the same type of fluidic sample against different growth media.

Accordingly, the spirit and scope of the present invention is intended to be limited only by the terms of the appended claims.

What is claimed is:

1. A test card for separately but simultaneously or sequentially detecting the existence and/or identity of different types of microorganisms, comprising:
a top layer having
a peripheral side strip along a side of the top layer,
a bottom surface covered with an adhesive film,
a first growth media covering the adhesive film on bottom surface,
except in the area of peripheral side strip,
a central layer having
a top surface,
a bottom surface,
a bottom layer having
a top surface,
a bottom surface covered with adhesive film,
a second growth media covering the adhesive film, except in the area of bottom layer corresponding to the location of peripheral side strip,
wherein the first growth media has a chemical formulation selected to facilitate the growth or detection of a first type of microorganism, and the second growth media has a chemical formulation selected to facilitate the growth or detection of a second type of microorganism and has a different chemical formulation than the chemical formulation of the first growth media.

2. The card according to claim 1 wherein the top layer and the bottom layer are each secured along a portion of their periphery to the central layer, and the top and bottom layers are formed from flexible material such that they can be moved away from the central layer sufficiently to allow deposition of a fluidic test sample on the central layer without complete removal of the top or bottom layers from the central layers.

3. The card according to claim 1 wherein the portion of top layer which is not covered by the first growth media is used to attach the top layer to the central layer, and that portion corresponds dimensionally with the peripheral side strip.

4. The card according to claim 1 wherein the central layer is opaque and waterproof, the top and bottom layers are gas permeable, flexible, and non-toxic to the microorganisms being tested for, and the adhesive film is gas permeable and non-toxic to the microorganisms being tested for.

5. The card according to claim 4 wherein the top layer and adhesive film of the top layer are transparent sufficiently to allow visual detection of color indicia resulting in the growth media.

6. The card according to claim 5 wherein the top layer and adhesive film of the top layer are transparent sufficiently to allow visual detection of both chromogenic and fluorogenic color indicia resulting in the growth media.

7. The card according to claim 5 wherein the top layer is formed from material which will retain ink in a grid pattern when it is applied to the top layer, and whereby that ink does not diffuse, smudge, or smear on the top layer during normal testing of the card.

8. The card according to claim 5 wherein the top layer is formed from material which will removably retain an ink grid pattern applied to the top layer.

9. The card according to claim 8 the bottom layer is formed from material which will removably retain ink applied to the bottom layer.

10. The card according to claim 9 wherein the central layer is formed from non-absorbent material, with respect to the chemistry of the fluidic test sample applied to the card.

11. The card according to claim 1 wherein
the top and bottom layers are formed from acetate film of 2 mil thickness, and
the central layer is formed from opaque artificial paper of 10 mill thickness.

12. A test card for separately but simultaneously or sequentially detecting the existence and/or identity of different samples of microorganisms, comprising:
a top layer having
a peripheral side strip along a side of the top layer,
a bottom surface covered with an adhesive film,
a first growth media covering the adhesive film on bottom surface, except in the area of peripheral side strip,
a central layer having
a top surface,
a bottom surface,
a bottom layer having
a top surface,
a bottom surface covered with adhesive film,
a second growth media covering the adhesive film, except in the area of bottom layer corresponding to the location of peripheral side strip,
wherein the first growth media has a chemical formulation selected to facilitate the growth or detection of a first sample of microorganism, and the second growth media has a chemical formulation selected to facilitate the growth or detection of a second sample of microorganism.
